(19) United States
(12) Patent Application Publication
(10) Pub. No.: US 2006/0193808 A1
(43) Pub. Date: Aug. 31, 2006

(54) Composition for coating keratin fibers, comprising a fatty alcohol wax and a cellulose-based polymer

(76) Inventor: Frederic Auguste, Chevilly-Larue (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/347,336

(22) Filed: Feb. 6, 2006

Related U.S. Application Data
(60) Provisional application No. 60/652,752, filed on Feb. 15, 2005.

(30) Foreign Application Priority Data
Feb. 4, 2005 (FR) 05 50337

(51) Int. Cl. A61K 8/73

(52) U.S. Cl. 424/70.13

(57) ABSTRACT

The present disclosure relates to a composition for coating keratin fibers, comprising, in a cosmetically acceptable aqueous medium, at least one fatty alcohol wax, and at least one cellulose-based polymer. The present disclosure further relates to the use of such a composition to obtain charging makeup for keratin fibers.

This application claims benefit of U.S. Provisional Application No. 60/652,752, filed Feb. 15, 2005, the contents of which are incorporated herein by reference. This application also claims benefit of priority under 35 U.S.C. § 119 to French Patent Application No. 05 50337, filed Feb. 4, 2005, the contents of which are also incorporated herein by reference.

The present disclosure relates to a cosmetic composition for coating keratin fibers, comprising a fatty alcohol wax and a cellulose-based polymer.

The composition according to the disclosure may, for example, be a makeup composition, such as a mascara, a makeup base for keratin fibers, a base coat, a composition to be applied over a makeup (also known as a top coat), or a composition for treating keratin fibers.

In one non-limiting embodiment of the present disclosure, the composition is a leave-in composition.

In another non-limiting embodiment of the present disclosure the composition is a mascara.

As used herein, the term “mascara” means a composition intended to be applied to the eyelashes: it may be an eyelash makeup composition, an eyelash makeup base, a composition to be applied over a mascara (also known as a top coat), or a cosmetic eyelash treatment composition. Mascara may be applied to both human eyelashes and false eyelashes.

Eyelash makeup compositions generally comprise a wax or a mixture of waxes that are dispersed using at least one surfactant in an aqueous phase that also contains polymers and pigments.

It is generally by means of the qualitative and quantitative choice of the waxes and polymers that the desired application specificities for makeup compositions are adjusted, for instance their fluidity, their covering power and/or their curling power. Thus, it is possible to produce various compositions, which, when applied, for example, to the eyelashes, induce a variety of effects such as lengthening, curling and/or thickening (charging effect).

The present disclosure is also directed towards a composition that is useful for producing a thick (also known as “charging”) makeup result on keratin fibers, such as the eyelashes. For the purposes of the present disclosure, the term “keratin fibers” includes the hair, the eyelashes and the eyebrows, as well as synthetic wigs and false eyelashes.

It is known in the art that the higher the solids content (provided in part by a fatty phase comprising, for example, at least one wax or at least one lipophilic polymer) in a composition, the greater will be the amount of material deposited on the eyelash, and thus the more volumizing will be the result obtained.

However, increasing the solids content in a composition, such as an emulsion or dispersion, may result in an increase in the consistency of the product obtained. As a result, application of the composition to the eyelashes may be intricate and difficult, because the product may be thick, viscous, difficult to deposit, and/or may deposit non-uniformly and/or in lumps. The makeup thus obtained may have a coarse, granular appearance, may not be uniform, and may look unattractive.

These problems are often found in “volumizing” mascaras, which may be difficult to apply and may give a non-uniform makeup.

Mascaras with a volumizing effect comprising an aqueous phase, a cellulose-based polymer and polar waxes such as carnauba wax and/or candelilla wax, and which are easy to apply and give a uniform deposit, are known in the art.

One aspect of the present disclosure is to propose another formulation route for a composition for coating keratin fibers leading to a charging effect on the keratin fibers, and which totally or partially solves the problems associated with the conventional formulation routes.

It has been found, unexpectedly, that it is possible to prepare compositions allowing thickening makeup of keratin fibers and a smooth and uniform deposit on the fibers by incorporating in these compositions a combination of a fatty alcohol wax and a cellulose-based polymer.

Accordingly, one aspect of the present disclosure is a composition for coating keratin fibers, comprising a cosmetically acceptable aqueous medium comprising at least 3% of least one fatty alcohol wax and a cellulose-based polymer.

Another aspect of the present disclosure is a composition for coating keratin fibers, comprising a cosmetically acceptable aqueous medium comprising at least one fatty alcohol wax, at least one cellulose-based polymer, and at least one anionic surfactant.

Yet another aspect of the present disclosure is a process for making up keratin fibers, wherein a composition in accordance with the disclosure is applied to said fibers.

The present disclosure also relates to the use of a composition in accordance with the disclosure to obtain charging makeup on keratin fibers, for example, the eyelashes and the eyebrows, and/or a smooth and uniform deposit on said fibers.

Another aspect of the present disclosure is the use of a combination of at least one fatty alcohol wax and of a cellulose-based polymer to obtain charging makeup on keratin fibers, such as the eyelashes and the eyebrows, and/or a smooth and uniform deposit on the fibers.

For the purposes of the present disclosure, the term “charging” means a thick and volumizing makeup on keratin fibers, for example, the eyelashes.

The compositions according to the present disclosure may, in at least one embodiment, have a solids content of greater than or equal to 40% by weight, for example, greater than or equal to 42% by weight, such as greater than or equal to 45%, for example, greater than or equal to 47% by weight, or up to 60% by weight, relative to the total weight of the composition.

The solids content, i.e. the content of non-volatile matter, may be measured in different ways. For example, the solids content may be measured by methods employing oven drying, methods employing drying by exposure to infrared radiation, as well as chemical methods employing Karl Fischer water titration.

For example, the amount of solids, commonly referred to as the “dry extract”, of the compositions according to the disclosure may be measured by heating a sample with infrared rays with a wavelength of from 2 μm to 3.5 μm. Substances contained in the sample that have a high vapor pressure evaporate under the effect of this radiation. By measuring the weight loss of the sample, the “dry extract” of the composition may be determined. These measurements may be performed using an LP 16 commercial infrared desiccator from Mettler. This technique is fully described in the machine documentation supplied by Mettler.

The protocol for measuring the dry extract of the composition is as follows:

About 1 gram of a sample composition is spread out on a metal crucible. This crucible, after being placed in the desiccator, is subjected to a nominal temperature of 120° C. for one hour. The wet mass of the sample, corresponding to the initial mass, and the dry mass of the sample, corresponding to the mass after exposure to the radiation, are measured using a precision balance.

The solids content is calculated in the following manner:
Dry extract=100×(dry mass/wet mass).
Fatty Alcohol Wax

For the purposes of the present disclosure, the term “wax” generally means a lipophilic compound that is solid at room temperature (25° C.), with a solid/liquid reversible change of state, having a melting point of greater than or equal to 30° C., which may be up to 120° C.

By bringing the wax to the liquid form (melting), it is possible to make it miscible with oils, and to form a microscopically uniform mixture, but on cooling the mixture to room temperature, recrystallization of the wax in the oils of the mixture is obtained.

Non-limiting examples of waxes that are suitable for the composition of the present disclosure include, but are not limited to, waxes having a melting point of greater than or equal to 45° C., including those having a melting point greater than or equal to 50° C., for example, those having a melting point greater than or equal to 55° C.

The melting point of the wax may be measured using a differential scanning calorimeter (DSC), such as the calorimeter sold under the name MDSC 2929 by the company TA Instruments.

The protocol for measuring the melting point of wax is as follows:

A sample of 5 mg of wax placed in a crucible is subjected to a first temperature rise ranging from 0° C. to 120° C., at a heating rate of 10° C./minute, is then cooled from 120° C. to 0° C. at a cooling rate of 10° C./minute and is finally subjected to a second temperature increase ranging from 0° C. to 120° C. at a heating rate of 5° C./minute. During the second temperature increase, the variation of the difference in power absorbed by the empty crucible and by the crucible containing the sample of product is measured as a function of the temperature. The melting point of the compound is the temperature value corresponding to the top of the peak of the curve representing the variation in the difference in absorbed power as a function of the temperature.

The fatty alcohol waxes may, for example, be chosen from saturated or unsaturated, branched or unbranched fatty alcohols containing from 20 to 60 carbon atoms, or mixtures comprising at least 30% of the fatty alcohols, for example with polyethylene.

The fatty alcohols may be linear and may comprise from 14 to 60, for example from 20 to 58, carbon atoms. In at least one non-limiting embodiment of the present disclosure, the fatty alcohols correspond to the following chemical formula:
CH₃—(CH₂)ₙ—CH₂OH
wherein n is an integer ranging from 16 to 58, for example, from 18 to 56.

In at least one non-limiting embodiment of the present disclosure, the fatty alcohols are chosen from branched fatty alcohols containing from 24 to 80 carbon atoms.

Non-limiting examples of C₂₀-C₆₀ fatty alcohols include, for example, fatty alcohols that are commercially available from the company New Phase Technologies under the names Performacol® 350, Performacol®7 425, Performacol® 550 and Performacol® 700, or from the company Petrolite under the names Unilin® 350 Alcohol, Unilin® 425 Alcohol, Unilin® 550 Alcohol and Unilin® 700 Alcohol. These are mixtures of very long chain linear alcohols that are obtained via a polymerization process that allows polymers with a very low polydispersity index to be obtained (Mw/Mn=1.1). Their weight-average molar mass is between about 350 and 1000.

Non-limiting mention may also be made of behenyl alcohol, cetyl alcohol and stearyl alcohol, and mixtures thereof.

The fatty alcohol may be present in the composition in an amount ranging from 3% to 50% by weight, for example, from 5% to 40% by weight, from 7% to 30% by weight, or from 10% to 30% by weight, relative to the total weight of the composition.

Additional Wax

The composition according to the disclosure may further comprise at least one additional wax.

The additional wax may, for example, be chosen from waxes of animal, plant, mineral or synthetic origin that are solid and rigid at room temperature, and mixtures thereof.

The additional wax may also have a hardness ranging from 0.05 MPa to 30 MPa. The hardness of the additional wax is determined by measuring the compression force, which is measured at 20° C. using the texturometer sold under the name TA-XT2 by the company Rheo, equipped with a stainless-steel cylinder 2 mm in diameter, travelling at a measuring speed of 0.1 mm/second, and penetrating into the wax to a penetration depth of 0.3 mm.

The protocol for measuring the hardness of the additional wax is as follows:

The wax is melted at a temperature equal to the melting point of the wax+10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C., after which the hardness or the tack of the wax is measured.

The texturometer spindle is displaced at a speed of 0.1 mm/s, and then penetrates the wax to a penetration depth of 0.3 mm. When the spindle has penetrated the wax to a depth of 0.3 mm, the spindle is held still for 1 second (corresponding to the relaxation time) and is then withdrawn at a speed of 0.5 mm/s.

The hardness value is the maximum compression force measured divided by the area of the texturometer cylinder in contact with the wax.

Non-limiting examples of additional waxes useful herein include hydrocarbon-based waxes, such as beeswax, lanolin wax, lemon wax, orange wax and Chinese insect waxes; rice bran wax, carnauba wax, candelilla wax, ouricury wax, Japan wax, berry wax, shellac wax and sumach wax; montan wax, microcrystalline waxes, paraffins and ozokerite; polyethylene wax, polymethylene wax, the waxes obtained by Fischer-Tropsch synthesis and waxy copolymers, and esters thereof.

Further non-limiting examples of additional waxes useful herein include waxes obtained by catalytic hydrogenation of animal or plant oils containing linear or branched C₈-C₃₂ fatty chains.

As examples of the aforementioned additional waxes, non-limiting mention may be made of hydrogenated jojoba oil, isomerized jojoba oil such as the trans-isomerized partially hydrogenated jojoba oil manufactured or sold by the company Desert Whale under the commercial name Iso-Jojoba-50®, hydrogenated sunflower oil, hydrogenated castor oil, hydrogenated coconut oil, hydrogenated lanolin oil and bis(1,1,1-trimethylolpropane)tetrastearate sold under the name “Hest 2T-4S” by the company Heterene and bis(1,1,1-trimethylolpropane)tetrabehenate sold under the name “Hest 2T-4B” by the company Heterene.

Non-limiting mention may also be made of silicone waxes and modified silicone waxes, such as silicone-treated candelilla wax, and fluoro waxes.

The wax obtained by hydrogenation of olive oil esterified with stearyl alcohol, sold under the name “Phytowax Olive 18 L57” or else the waxes obtained by hydrogenation of castor oil esterified with cetyl alcohol sold under the names “Phytowax ricin 16L64 and 22L73” by the company Sophim may also be used. Such waxes are described in French published patent application FR-A-2 792 190.

According to at least one non-limiting embodiment of the present disclosure, the additional wax has a hardness of less than or equal to 4 MPa, for example, less than or equal to 3.5 MPa.

Non-limiting examples of such waxes include oxypropylenated lanolin wax (5 EO), orange wax, lemon wax, hydrogenated castor oil wax, the mixture of esters of aliphatic acids and of primary alcohols sold under the name Burco LB-02, the olive wax (unsaponifiable matter of hydrogenated olive oil) sold under the name Inholive by the company Exa International, the PDMS-grafted behenyl methacrylate wax sold under the name KP-562 by the company Shin-Etsu, fluoropolymethylalkyldimethylsiloxane wax such as the product sold under the name Wax 23087 by the company Wacker, C30-C45 alkyl dimethicone wax such as the product sold under the name SF 1642 by GE Bayer, ethoxylated bis(trimethylolpropane)tetrastearate (5 EO), and certain paraffin waxes, such as the wax sold under the name Cerafine 56/58 by the company Baerlocher.

According to at least one non-limiting embodiment of the present disclosure, the additional wax is a “tacky” wax, i.e. it has a tack of greater than or equal to 0.1 N·s.

Non-limiting examples of “tacky” waxes that may be utilized according to the present disclosure include those having a tack ranging from 0.1 N·s to 10 N·s, for example, from 0.1 N·s to 5 N·s, such as from 0.2 N·s to 5 N·s, for example, from 0.3 N·s to 2 N·s.

The tack of the wax is determined by measuring the change in the force (compression force) as a function of time, at 20° C., according to the protocol indicated above for hardness.

During the 1-second relaxation time, the force (compression force) decreases greatly until it becomes zero, and then, during the withdrawal of the spindle, the force (stretching force) becomes negative and then rises again to the value 0. The tack of the wax corresponds to the integral of the curve of the force as a function of time for the part of the curve corresponding to negative values of the force. The tack value is expressed in N·s.

In at least one non-limiting embodiment of the present disclosure, the tacky wax has a hardness of less than or equal to 3.5 MPa, such as from 0.01 MPa to 3.5 MPa, for example, from 0.05 MPa to 3 MPa.

Hardness is measured according to the protocol described above.

Non-limiting examples of tacky waxes that may be used in the compoition of the present disclosure include C₂₀-C₄₀ alkyl(hydroxystearyloxy)stearates, wherein the alkyl group contains from 20 to 40 carbon atoms, either alone or as a mixture.

Non-limiting examples of such tacky waxes include the waxes sold under the names “Kester Wax K 82 P®” and “Kester Wax K 80 P®” by the company Koster Keunen.

The composition according to the present disclosure may comprise a total content of waxes (fatty alcohol wax and additional wax(es)) ranging from 1% to 50%, from 5% to 40%, from 10% to 35%, or from 10% to 30% by weight, relative to the total weight of the composition.

The wax(es) (including the tacky wax) may be present in the composition in the form of an aqueous microdispersion of wax. As used herein, the expression “aqueous microdispersion of wax” means an aqueous dispersion of wax particles in which the size of the wax particles is less than or equal to about 1 μm.

Wax microdispersions are stable dispersions of colloidal wax particles, and are described, for instance, in “Microemulsions Theory and Practice”, L. M. Prince Ed., Academic Press (1977) pages 21-32.

The aforementioned wax microdispersions may, for example, be obtained by melting the wax in the presence of a surfactant, and optionally of a portion of water followed by the gradual addition of hot water with stirring. The intermediate formation of a water-in-oil emulsion is observed, followed by a phase inversion, with final production of a oil-in-water microemulsion. On cooling, a stable microdispersion of solid wax colloidal particles is obtained.

These wax microdispersions may also be obtained by stirring the mixture of wax, surfactant and water using stirring means such as ultrasound, high-pressure homogenizers or turbomixers.

The particles of the wax microdispersion may, for example, have a mean size of less than 1 μm, such as less than 0.5 μm. In at least one non-limiting embodiment of the present disclosure, the particles of the wax microdispersion have a mean particle size ranging from 0.02 μm to 0.99 μm. In another non-limiting embodiment of the present disclosure, the particles of the wax microdispersion have a mean particle size ranging from 0.06 μm to 0.5 μm.

These particles mainly comprise a wax or a mixture of waxes. However, they may also comprise a small proportion of oily and/or pasty fatty additives, a surfactant and/or a common liposoluble additive/active agent.

When the wax or the mixture of waxes is present in the composition according to the present disclosure in the form of an aqueous dispersion of particles, the size of the particles, expressed as the mean “effective” volume diameter D[4,3] as defined hereinbelow, may be, for example, less than or equal to 1 μm, such as less than or equal to 0.75 μm.

The wax particles may have varied shapes. In at least one non-limiting embodiment, the wax particles may be spherical.

Cellulose-Based Polymer

In at least one non-limiting embodiment of the present disclosure, the cellulose-based polymer is film-forming. As used herein, the term “film-forming” means a polymer capable, by itself or in the presence of an auxiliary film-forming agent, of forming a continuous film that adheres to a support and especially to keratin materials, for example, a cohesive film, such as a film whose cohesion and mechanical properties are such that the film may be isolated from the support.

The term “cellulose-based polymer,” as used herein, means cellulose or a cellulose derivative.

The cellulose-based polymer may be chosen, for example, from alkylcelluloses such as methylcellulose, hydroxyalkylcelluloses such as hydroxyethylcellulose, hydroxypropylcellulose or ethylhydroxyethylcellulose, and carboxyalkylcelluloses such as carboxymethylcellulose, and mixtures thereof.

The cellulose-based polymer may be present in the composition according to the present disclosure in a solids content ranging from 0.1% to 30% by weight, for example, from 0.5% to 20% by weight, such as from 1% to 15% by weight, relative to the total weight of the composition.

The composition according to the present disclosure may further comprise, in addition to the cellulose-based polymer, an additional film-forming polymer.

The additional film-forming polymer may be present in the composition according to the disclosure in a solids content ranging from 0.1% to 60% by weight, for example, from 0.5% to 40% by weight, such as from 1% to 30% by weight, relative to the total weight of the composition.

Among the film-forming polymers that may be used in the composition of the present disclosure, non-limiting mention may be made of synthetic polymers, of free-radical type or of polycondensate type, and polymers of natural origin, and mixtures thereof.

The expression “free-radical film-forming polymer,” as used herein, means a polymer obtained by polymerization of unsaturated monomers, in particular ethylenically unsaturated monomers, each monomer being capable of homopolymerizing (unlike polycondensates).

The film-forming polymers of free-radical type may be, for example, vinyl polymers or copolymers, including acrylic polymers.

The vinyl film-forming polymers may be obtained by the polymerization of monomers containing ethylenic unsaturation and containing at least one acidic group and/or esters of these acidic monomers and/or amides of these acidic monomers.

Non-limiting examples of monomers bearing an acidic group that may be used include α,β-ethylenic unsaturated carboxylic acids, such as acrylic acid, methacrylic acid, crotonic acid, maleic acid or itaconic acid. In at least one non-limiting embodiment, the monomer bearing an acidic group is chosen from (meth)acrylic acid and crotonic acid. In a further non-limiting embodiment, the monomer bearing an acidic group is (meth)acrylic acid.

The esters of acidic monomers may be chosen, for example, from (meth)acrylic acid esters (also known as (meth)acrylates), including alkyl(meth)acrylates, such as C₁-C₃₀ or C₁-C₂₀ alkyl(methacrylates), aryl(meth)acrylates, such as C₆-C₁₀ aryl(meth)acrylates, and hydroxyalkyl(meth)acrylates, such as C₂-C₆ hydroxyalkyl(methacrylates).

Among the alkyl(meth)acrylates, non-limiting mention is made of methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl(meth)acrylates, non-limiting mention is made of hydroxyethyl acrylate, 2-hydroxypropyl acrylate, hydroxyethyl methacrylate and 2-hydroxy-propyl methacrylate.

Among the aryl(meth)acrylates, non-limiting mention is made of benzyl acrylate and phenyl acrylate.

In at least one non-limiting embodiment of the present disclosure, the (meth)acrylic acid esters are chosen from alkyl(meth)acrylates.

According to at least one non-limiting embodiment of the present disclosure, the alkyl group of the esters may be either fluorinated or perfluorinated, i.e. some or all of the hydrogen atoms of the alkyl group are substituted with fluorine atoms.

Non-limiting examples of amides of the acid monomers that may be mentioned include (meth)acrylamides, including N-alkyl(meth)acrylamides, such as C₂-C₁₂ alkyl(meth)acrylamides. Among the N-alkyl(meth)acrylamides, non-limiting mention is made of N-ethylacrylamide, N-t-butylacrylamide, N-t-octylacrylamide and N-undecylacrylamide.

The vinyl film-forming polymers may also be obtained from the homopolymerization or copolymerization of monomers chosen from vinyl esters and styrene monomers. For example, these monomers may be polymerized with acid monomers and/or esters thereof and/or amides thereof, such as those mentioned above.

Non-limiting examples of vinyl esters that may be mentioned include vinyl acetate, vinyl neodecanoate, vinyl pivalate, vinyl benzoate and vinyl t-butylbenzoate. Non-limiting examples of styrene monomers that may be mentioned include styrene and α-methylstyrene.

Among the film-forming polycondensates, non-limiting mention is made of polyurethanes, polyesters, polyesteramides, polyamides, epoxyester resins and polyureas.

The polyurethanes may be chosen, for example, from anionic, cationic, nonionic and amphoteric polyurethanes, polyurethane-acrylics, polyurethane-polyvinyl-pyrrolidones, polyester-polyurethanes, polyether-polyurethanes, polyureas and polyurea/polyurethanes, and mixtures thereof.

The polyesters may be obtained, for example, by polycondensation of dicarboxylic acids with polyols, in particular diols.

The dicarboxylic acid may be aliphatic, alicyclic or aromatic. Non-limiting examples of such acids include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers may be used alone or as a combination of at least two dicarboxylic acid monomers. Among these monomers, non-limiting mention is made of phthalic acid, isophthalic acid and terephthalic acid.

The diol may be chosen from aliphatic, alicyclic and aromatic diols. Non-limiting examples of such diols include: ethylene glycol, propylene glycol, diethylene glycol, triethylene glycol, 1,3-propanediol, cyclohexanedimethanol and 4-butanediol. Other non-limiting examples of polyols that may be used include glycerol, pentaerythritol, sorbitol and trimethylolpropane.

The polyesteramides may be obtained in a manner analogous to that of the polyesters, namely by the polycondensation of diacids with diamines or amino alcohols. Non-limiting examples of diamines that may be used include: ethylenediamine, hexamethylenediamine and meta- or para-phenylenediamine. A non-limiting example of an amino alcohol that may be used is monoethanolamine.

The polyester may also comprise at least one monomer bearing at least one group —SO₃M, in which M represents a hydrogen atom, an ammonium ion NH₄⁺ or a metal ion such as, for example, an Na⁺, Li⁺, K⁺, Mg²⁺, Ca²⁺, Cu²⁺, Fe²⁺ or Fe³⁺ ion. In at least one non-limiting embodiment of the present disclosure, a difunctional aromatic monomer comprising such a group —SO₃M may be used.

The aromatic nucleus of the difunctional aromatic monomer also bearing a group —SO₃M as described above may be chosen, for example, from benzene, naphthalene, anthracene, biphenyl, oxybiphenyl, sulfonylbiphenyl and methylenebiphenyl nuclei. As examples of difunctional aromatic monomers also bearing a group —SO₃M, non-limiting mention may be made of: sulfoisophthalic acid, sulfoterephthalic acid, sulfophthalic acid, and 4-sulfonaphthalene-2,7-dicarboxylic acid.

In at least one non-limiting embodiment of the present disclosure, the copolymers are chosen from those based on isophthalate/sulfoisophthalate, for example, those copolymers obtained by condensation of diethylene glycol, cyclohexanedimethanol, isophthalic acid and sulfoisophthalic acid.

The composition may also comprise at least one water-soluble film-forming polymer chosen from, for example:

proteins, including, but not limited to, proteins of plant origin, such as wheat or soybean proteins; proteins of animal origin, such as keratins, for example keratin hydrolysates and sulfonic keratins;

anionic, cationic, amphoteric or nonionic chitin or chitosan polymers;

vinyl polymers, including, for instance, polyvinylpyrrolidones, copolymers of methyl vinyl ether and of maleic anhydride, the copolymer of vinyl acetate and of crotonic acid, copolymers of vinylpyrrolidone and of vinyl acetate; copolymers of vinylpyrrolidone and of caprolactam; and polyvinyl alcohol;

polymers of natural origin, optionally modified, such as:
gum arabics, guar gum, xanthan derivatives and karaya gum;
alginates and carrageenans;
glycoaminoglycans, and hyaluronic acid and its derivatives;
shellac resin, sandarac gum, dammar resins, elemi gums and copal resins;
deoxyribonucleic acid;
mucopolysaccharides such as hyaluronic acid and chondroitin sulphate;

and mixtures thereof.

According to at least one embodiment of the composition according to the present disclosure, the film-forming polymer may be chosen from polymers dissolved in a liquid organic medium of the composition comprising oils or organic solvents, such as those described below (in which case the film-forming polymer is said to be a liposoluble polymer).

Non-limiting examples of liposoluble polymers that may be mentioned include copolymers of a vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer, which may be a vinyl ester (different from the vinyl ester already present), an α-olefin (containing from 8 to 28 carbon atoms), an alkyl vinyl ether (the alkyl group of which contains from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked using vinylic-, allylic-, or methallylic-type crosslinking agents, such as tetraallyloxyethane, divinylbenzene, divinyl octanedioate, divinyl dodecanedioate and divinyl octadecanedioate.

Non-limiting examples of these copolymers include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethyl-propionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Non-limiting examples of liposoluble film-forming polymers that may also be mentioned include liposoluble copolymers, such as those resulting from the copolymerization of vinyl esters containing from 9 to 22 carbon atoms or of alkyl acrylates or methacrylates, and alkyl radicals containing from 10 to 20 carbon atoms.

Such liposoluble copolymers may be chosen from, for example, polyvinyl stearate, polyvinyl stearate crosslinked with the aid of divinylbenzene, of diallyl ether or of diallyl phthalate copolymers, polystearyl(meth)acrylate, polyvinyl laurate and polylauryl(meth)acrylate copolymers, it being possible for these poly(meth)acrylates to be crosslinked with the aid of ethylene glycol dimethacrylate or tetraethylene glycol dimethacrylate.

The liposoluble copolymers defined above are described in, for example, published French patent application FR-A-2 232 303, and may have a weight-average molecular weight ranging from 2000 to 500 000, such as from 4000 to 200 000.

As liposoluble film-forming polymers that may be used in the present disclosure, non-limiting mention may also be made of polyalkylenes, such as copolymers of C₂-C₂₀ alkenes, for example, polybutene; alkylcelluloses with a linear or branched, saturated or unsaturated C₁-C₈ alkyl radical, such as ethylcellulose and propylcellulose; copolymers of vinylpyrrolidone (VP), such as copolymers of vinylpyrrolidone and of a C₂ to C₄₀ or C₃ to C₂₀ alkene. As examples of VP copolymers which may be used in the present disclosure, non-limiting mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

The film-forming polymer may also be present in the composition in the form of particles dispersed in an aqueous phase or in a non-aqueous solvent phase (liquid organic medium of the composition), which is generally known as a latex or pseudolatex. The techniques for preparing these dispersions are well known to those skilled in the art.

Non-limiting examples of aqueous dispersions of film-forming polymers that may be used in the present disclosure include the acrylic dispersions sold under the names “Neocryl XK-90®”, “Neocryl A-1070®”, “Neocryl A-1090®”, “Neocryl BT-62®”, “Neocryl A-1079®” and “Neocryl A-523®” by the company Avecia-Neoresins, “Dow Latex 432®” by the company Dow Chemical, “Daitosol 5000 AD®” or “Daitosol 5000 SJ®” by the company Daito Kasey Kogyo; “Syntran 5760®” by the company Interpolymer, the aqueous dispersions of polyurethane sold under the names “Neorez R-981®” and “Neorez R-974®” by the company Avecia-Neoresins, “Avalure UR-405®”, “Avalure UR-410®”, “Avalure UR-425®”, “Avalure UR-450®”, “Sancure 875®”, “Sancure 861®”, “Sancure 878®” and “Sancure 2060®” by the company Goodrich, “Impranil 85®” by the company Bayer and “Aquamere H-1511®” by the company Hydromer; the sulfopolyesters sold under the brand name “Eastman AQ®” by the company Eastman Chemical Products, and vinyl dispersions, for instance “Mexomer PAM®” by the company Chimex, and mixtures thereof.

The composition according to the disclosure may also comprise a plasticizer that promotes the formation of a film with the film-forming polymer. Such a plasticizer may be chosen from any compound known to those skilled in the art as being capable of satisfying the desired function.

Cosmetically Acceptable Aqueous Medium

The cosmetically acceptable aqueous medium of the composition according to the disclosure may consist essentially of water or it may comprise a mixture of water and of water-miscible solvent (water miscibility of greater than 50% by weight at 25° C.). Non-limiting examples of such water-miscible solvents include lower monoalcohols containing from 1 to 5 carbon atoms, such as ethanol and isopropanol, glycols containing from 2 to 8 carbon atoms, such as propylene glycol, ethylene glycol, 1,3-butylene glycol and dipropylene glycol, C₃-C₄ ketones and C₂-C₄ aldehydes, and mixtures thereof.

The aqueous medium (water and, optionally, the water-miscible solvent) may be present in the composition of the present disclosure in an amount ranging from 0.1% to 95% by weight, for example, from 1% to 80% by weight, relative to the total weight of the composition.

Emulsifying System

According to the present disclosure, an emulsifying system chosen in an appropriate manner to obtain an oil-in-water or wax-in-water emulsion is used.

The compositions of the present disclosure may contain emulsifying surfactants. These emulsifying surfactants may be present in the composition in an amount ranging from 0.1% to 40% by weight, for example, from 0.3% to 20% by weight, relative to the total weight of the composition.

The ionic surfactants used in the context of the present disclosure may be anionic, cationic, nonionic or amphoteric. In at least one non-limiting embodiment, an anionic surfactant is used.

As examples of anionic surfactants that are suitable for use in the composition of the present disclosure, non-limiting mention may be made of:

C₁₆-C₃₀ fatty acid salts, including, for example, those derived from amines, such as triethanolamine stearate and/or 2-amino-2-methyl-1,3-propanediol stearate;

polyoxyethylenated fatty acid salts, including, for example, those derived from amines or alkali metal salts, and mixtures thereof;

phosphoric esters and salts thereof, such as “DEA oleth-10 phosphate” (Crodafos N 10N from the company Croda);

sulfosuccinates such as “Disodium PEG-5 citrate lauryl sulfosuccinate” and “Disodium ricinoleamido MEA sulfosuccinate”;

alkyl ether sulfates, such as sodium lauryl ether sulfate;

isethionates;

acylglutamates, such as “Disodium hydrogenated tallow glutamate” (Amisoft HS-21 R sold by the company Ajinomoto), and mixtures thereof.

In at least one non-limiting embodiment of the present disclosure, the at least one anionic surfactant is chosen from triethanolamine stearate and/or 2-amino-2-methyl-1,3-propanediol stearate. These surfactants may be obtained by simple mixing of stearic acid with triethanolamine and/or with 2-amino-2-methyl-1,3-propanediol.

The anionic surfactant(s) of the present disclosure may be present in the composition in an amount ranging from 0.1% to 30% by weight, for example, from 0.5% to 20% by weight, such as from 1% to 15% by weight, relative to the total weight of the composition.

Non-limiting examples of cationic surfactants include:

alkylimidazolidiniums, such as isostearylethylimidonium ethosulfate,

ammonium salts, such as N,N,N-trimethyl-1-docosanaminium chloride (behentrimonium chloride).

The composition according to the present disclosure may further comprise at least one nonionic or amphoteric surfactant. Reference may be made to the document “Encyclopedia of Chemical Technology, Kirk-Othmer”, volume 22, pp. 333-432, 3rd edition, 1979, Wiley, for the definition of the properties and (emulsifying) functions of surfactants, in particular pp. 347-377 of this reference, for anionic, amphoteric and nonionic surfactants.

Fatty Phase

For the purposes of the present disclosure, the term “fatty phase” means a phase composed of one or more non-aqueous substances that are liquid or solid at room temperature (25° C.), which are generally mutually compatible, such as waxes, pasty fatty substances, oils and oils thickened with a structuring agent, and mixtures thereof. The surfactants as described above do not form part of the fatty phase.

Thus, the fatty phase of the composition according to the disclosure comprises at least one fatty alcohol wax and optionally an additional wax as described above.

The fatty phase may have a hardness of less than or equal to 6 MPa, for example, ranging from 0.5 to 6 MPa, such as from 0.7 to 4 MPa, for example, from 0.8 to 3 MPa.

The fatty phase may be present in the composition of the present disclosure in an amount ranging from 5% to 60% by weight, for example, from 10% to 50% by weight, such as from 15% to 40% by weight, relative to the total weight of the composition.

Oils

The composition according to the disclosure may comprise at least one oil.

The oil may be chosen from, for example, volatile oils and/or non-volatile oils, and mixtures thereof. In at least one non-limiting embodiment, the composition may comprise at least one volatile oil.

The oil may be present in the composition according to the present disclosure in an amount ranging from 0.1% to 60% by weight, for example, from 0.1% to 30% by weight, relative to the total weight of the composition.

For the purposes of the present disclosure, the term “volatile oil” means an oil that is capable of evaporating on contact with the skin or the keratin fiber in less than one hour, at room temperature and atmospheric pressure. The volatile organic solvent(s) and volatile oils of the disclosure are volatile organic solvents and cosmetic oils that are liquid at room temperature, with a non-zero vapor pressure at room temperature and atmospheric pressure, ranging, for example, from 0.13 Pa to 40 000 Pa (10⁻³ to 300 mmHg), from 1.3 Pa to 13 000 Pa (0.01 to 100 mmHg), or from 1.3 Pa to 1300 Pa (0.01 to 10 mmHg). As used herein, the term “non-volatile oil” means an oil that remains on the skin or the keratin fiber at room temperature and atmospheric pressure for at least several hours and which has a vapor pressure of less than 10⁻³ mmHg (0.13 Pa).

These oils may be chosen from, for example, hydrocarbon-based oils, silicone oils or fluoro oils, or mixtures thereof.

The term “hydrocarbon-based oil,” as used herein, means an oil mainly containing hydrogen and carbon atoms and optionally oxygen, nitrogen, sulfur or phosphorus atoms. The volatile hydrocarbon-based oils may be chosen from hydrocarbon-based oils containing from 8 to 16 carbon atoms, such as C₈-C₁₆ alkanes, including C₈-C₁₆ isoalkanes of petroleum origin (also known as isoparaffins), for example isododecane (also known as 2,2,4,4,6-pentamethylheptane), isodecane and isohexadecane, for example the oils sold under the trade names Isopar or Permethyl, branched C8-C16 esters and isohexyl neopentanoate, and mixtures thereof. Other volatile hydrocarbon-based oils, such as petroleum distillates, including those sold under the name Shell Solt by the company Shell, may also be used. In at least one non-limiting embodiment, the volatile solvent is chosen from volatile hydrocarbon-based oils containing from 8 to 16 carbon atoms, and mixtures thereof.

Non-limiting examples of volatile oils that may also be used in the composition of the present disclosure include volatile silicones, for instance volatile linear or cyclic silicone oils, including those with a viscosity ≦8 centistokes (8×10⁻⁶ m²/s) and/or containing from 2 to 7 silicon atoms, wherein these silicones optionally comprise alkyl or alkoxy groups containing from 1 to 10 carbon atoms. As volatile silicone oils that may be used in the disclosure, non-limiting mention is made of octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane and dodecamethyl pentasiloxane, and mixtures thereof.

Non-limiting mention is also made of the linear volatile alkyltrisiloxane oils of general formula (I): 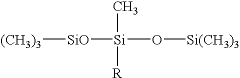
wherein R represents an alkyl group containing from 2 to 4 carbon atoms, wherein at least one hydrogen atom on said carbon atoms may be substituted with at least one fluorine or chlorine atom.

Among the oils of general formula (I), non-limiting mention is made of:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is, respectively, a butyl group, a propyl group or an ethyl group.

Volatile fluorinated solvents such as nonafluoromethoxybutane or perfluoromethylcyclopentane may also be used.

The composition according to the disclosure may also comprise at least one non-volatile oil. The at least one non-volatile oil may be chosen from, for example, non-volatile hydrocarbon-based oils and/or silicone oils and/or fluoro oils.

As examples of such non-volatile hydrocarbon-based oils, non-limiting mention is made of:

hydrocarbon-based oils of plant origin, such as triesters of fatty acids and of glycerol, the fatty acids of which may have varied chain lengths from C4 to C24, these chains possibly being linear or branched, and saturated or unsaturated; these oils may be, for example wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; or caprylic/capric acid triglycerides, for example, those sold by the company Stéarineries Dubois or those sold under the names Miglyol 810, 812 and 818 by the company Dynamit Nobel,

synthetic ethers containing from 10 to 40 carbon atoms,

linear or branched hydrocarbons of mineral or synthetic origin, such as petroleum jelly, polydecenes, hydrogenated polyisobutene such as parleam, and squalane, and mixtures thereof,

synthetic esters, such as oils of the formula R₁COOR₁, in which R₁ is chosen from linear or branched fatty acid residues containing from 1 to 40 carbon atoms, and R₁ is chosen from optionally branched, C₁-C₄₀ hydrocarbon-based chains, with the proviso that R1+R2≧10. Examples of such synthetic esters include, but are not limited to purcellin oil (cetostearyl octanoate), isopropyl myristate, isopropyl palmitate, C12 to C15 alkyl benzoates, hexyl laurate, diisopropyl adipate, isononyl isononanoate, 2-ethylhexyl palmitate, isostearyl isostearate, alcohol or polyalcohol octanoates, decanoates or ricinoleates, such as propylene glycol dioctanoate; hydroxylated esters, such as isostearyl lactate or diisostearyl malate; and pentaerythritol esters,

fatty alcohols that are liquid at room temperature with a branched and/or unsaturated carbon-based chain containing from 12 to 26 carbon atoms, such as octyidodecanol, isostearyl alcohol, oleyl alcohol, 2-hexyldecanol, 2-butyloctanol and 2-undecylpentadecanol,

higher fatty acids such as oleic acid, linoleic acid or linolenic acid,

carbonates,

acetals,

citrates,

and mixtures thereof.

Non-limiting examples of non-volatile silicone oils that may be used in the composition according to the present disclosure include non-volatile polydimethylsiloxanes (PDMS), polydimethylsiloxanes comprising alkyl or alkoxy groups which are pendent and/or at the end of a silicone chain, wherein these alkyl or alkoxy groups each contain from 2 to 24 carbon atoms, phenyl silicones, such as phenyl trimethicones, phenyl dimethicones, phenyltrimethylsiloxydiphenylsiloxanes, diphenyl dimethicones, diphenylmethyldiphenyltrisiloxanes and 2-phenylethyltrimethylsiloxysilicates.

Non-limiting examples of fluoro oils that may be used in the present disclosure include fluorosilicone oils, fluoro polyethers and fluorosilicones, as described in published European application EP-A-847 752.

Structuring Agent

The composition of the present disclosure may further include at least one oil-structuring agent. This oil-structuring agent, if present, may be chosen from semi-crystalline polymers and lipophilic gelling agents, and mixtures thereof.

As used herein, the term “polymer” means compounds containing at least two repeating units, for example, at least three repeating units, including those having at least ten repeating units. Further, as used herein, the term, “semi-crystalline polymer,” refers to polymers comprising a crystallizable portion, a crystallizable side chain or a crystallizable block in the skeleton, an amorphous portion in the skeleton, which have a first-order reversible phase-change temperature, such as a solid-liquid transition (melting). When the crystallizable portion is in the form of a crystallizable block of the polymer skeleton, the amorphous portion of the polymer is in the form of an amorphous block. In this case, the semi-crystalline polymer is a block copolymer, for example, of the diblock, triblock or multiblock type, comprising at least one crystallizable block and at least one amorphous block. As used herein, the term “block” generally means at least five identical repeating units. The crystallizable block(s) is (are) of chemical nature different from that of the amorphous block(s).

The semi-crystalline polymer may have a melting point of greater than or equal to 30° C., for example, from 30° C. to 80° C., such as from 30° C. to 60° C. This melting point is a first-order change of state temperature.

The melting point may be measured by any known method, for example, by a method using a differential scanning calorimeter (DSC).

The semi-crystalline polymer(s) to which the disclosure applies may have a number-average molecular mass of greater than or equal to 1000. For example, the semi-crystalline polymer(s) of the composition of the disclosure may have a number-average molecular mass \overline{M}n ranging from 2000 to 800 000, from 3000 to 500 000, from 4000 to 150 000, these ranges where the upper limit is 100 000, for example from 4000 to 99 000. In at least one non-limiting embodiment of the present disclosure, the semi-crystalline polymer(s) utilized in the present disclosure have a number-average molecular mass of greater than 5600, for example ranging from 5700 to 99 000.

For the purposes of the present disclosure, the term “crystallizable chain or block” means a chain or block which, if it were alone, would reversibly change from the amorphous state to the crystalline state, depending on whether the system is above or below the melting point. For the purposes of the present disclosure, a “chain,” is a group of atoms, which is pendent or lateral relative to the polymer skeleton. A “block” is a group of atoms belonging to the skeleton, and constitutes one of the repeating units of the polymer. In at least one non-limiting embodiment of the present disclosure, the “crystallizable side chain” may be a chain containing at least six carbon atoms.

The semi-crystalline polymer may, for example, be chosen from block copolymers comprising at least one crystallizable block and at least one amorphous block, and homopolymers and copolymers bearing at least one crystallizable side chain per repeating unit, and mixtures thereof.

Such polymers are described, for example, in published European patent application EP 1 396 259.

According to a non-limiting embodiment of the disclosure, the polymer is derived from a monomer containing a crystallizable chain chosen from saturated C₁₄ to C₂₂ alkyl(meth)acrylates.

As an example of a semi-crystalline polymer that may be used in the composition according to the disclosure, non-limiting mention may be made of the Intelimer® products from the company Landec described in the brochure “Intelimer® polymers”, Landec IP22 (Rev. 4-97). These polymers are in solid form at room temperature (25° C.) and bear crystallizable side chains.

Gelling agents that may be used in the compositions according to the present disclosure may be organic or mineral, polymeric or molecular lipophilic gelling agents.

Non-limiting examples of mineral lipophilic gelling agents that may be mentioned include optionally modified clays, such as hectorites modified with a C₁₀ to C₂₂ fatty acid ammonium chloride, hectorite modified with distearyidimethylammonium chloride, and the product sold under the name Bentone 38V® by the company Elementis.

Non-limiting mention may also be made of fumed silica that is optionally subjected to a hydrophobic surface treatment, and having a particle size of less than 1 μm. For example, the surface of the silica may be chemically modfified by a chemical reaction that generates a reduced number of silanol groups at the surface of the silica. Further, silanol groups present in the silica may be substituted with hydrophobic groups, resulting in a hydrophobic silica. Non-limiting examples of these hydrophobic groups include:

trimethylsiloxyl groups, including those that are obtained by treating fumed silica in the presence of hexamethyldisilazane. These silicas are known as “silica silylate” according to the CTFA (6th edition, 1995), and are sold, for example, under the names Aerosil R812® by the company Degussa, and Cab-O-Sil TS-530® by the company Cabot;

dimethylsilyloxyl or polydimethylsiloxane groups, including those that are obtained by treating fumed silica in the presence of polydimethylsiloxane or dimethyldichlorosilane. These silicas are known as “silica dimethyl silylate” according to the CTFA (6th edition, 1995), and are sold, for example, under the names Aerosil R972® and Aerosil R974® by the company Degussa, and Cab-O-Sil TS-610® and Cab-O-Sil TS-720® by the company Cabot.

The hydrophobic fumed silica used herein may have a particle size that may be nanometric or micrometric. For example, the hydrophobic fumed silica of the present disclosure may, for example, have a particle size ranging from about 5 to 200 nm.

Non-limiting examples of the polymeric organic lipophilic gelling agents useful herein include, partially or totally crosslinked elastomeric organopolysiloxanes of three-dimensional structure, such as those sold under the names KSG6®, KSG16® and KSG18® from Shin-Etsu, Trefil E-505C® or Trefil E-506C® from Dow Corning, Gransil SR-CYC®, SR DMF 10®, SR-DC556®, SR 5CYC gel®, SR DMF 10 gel® and SR DC 556 gel® from Grant Industries and SF 1204® and JK 113® from General Electric; ethylcellulose, such as the product sold under the name Ethocel® by Dow Chemical; polycondensates of polyamide type resulting from condensation between (α) at least one acid chosen from dicarboxylic acids containing at least 32 carbon atoms, such as fatty acid dimers, and (β) an alkylenediamine and in particular ethylenediamine, in which the polyamide polymer comprises at least one carboxylic acid end group esterified or amidated with at least one saturated and linear monoalcohol or one saturated and linear monoamine containing from 12 to 30 carbon atoms, for example, ethylenediamine/stearyl dimer dilinoleate and ethylenediamine/stearyl dimer tallate copolymers such as the products sold under the name Uniclear® by the company Arizona Chemical; galactomannans comprising from one to six, for example from two to four hydroxyl groups per saccharide, and substituted with a saturated or unsaturated alkyl chain, such as guar gum alkylated with at least one C₁ to C₆, for example, a C₁ to C₃, alkyl chains, and mixtures thereof. Block copolymers of “diblock”, “triblock” or “radial” type, of the polystyrene/polyisoprene or polystyrene/polybutadiene type, such as the products sold under the name Luvitol HSB® by the company BASF, of the polystyrene/copoly(ethylene-propylene) type, such as the products sold under the name Kraton® by the company Shell Chemical Co., or of the polystyrene/copoly(ethylene-butylene) type, and mixtures of triblock and radial (star) copolymers in isododecane, such as those sold by the company Penreco under the name Versagel®, for example, the mixture of butylene/ethylene/styrene triblock copolymer and of ethylene/propylene/styrene star copolymer in isododecane (Versagel M 5960).

Silicone polyamides of the polyorganosiloxane type may also be used, such as those described in U.S. Pat. Nos. 5,874,069, 5,919,441, 6,051,216 and 5,981,680.

These silicone polymers may belong to the following two families:

polyorganosiloxanes comprising at least two groups capable of establishing hydrogen interactions, these two groups being located in the polymer chain, and/or

polyorganosiloxanes comprising at least two groups capable of establishing hydrogen interactions, these two groups being located on grafts or branches.

Among the lipophilic gelling agents that may be used in the compositions according to the present disclosure, non-limiting mention is made of fatty acid esters of dextrin, such as dextrin palmitates, e.g., the products sold under the name Rheopearl TL® or Rheopearl KL® by the company Chiba Flour.

Additives

The composition according to the present disclosure may also comprise at least one dyestuff, such as, for example, pulverulent dyestuffs, liposoluble dyes and water-soluble dyes.

The at least one dyestuff may be present in the composition of the present disclosure in an amount ranging from 0.01% to 30% by weight, relative to the total weight of the composition.

The pulverulent dyestuffs may be chosen from pigments and nacres. The pigments may be, for example, white or colored, mineral and/or organic, and coated or uncoated. Among the mineral pigments, non-limiting mention may be made of titanium dioxide, optionally surface-treated, zirconium oxide, zinc oxide, cerium oxide, iron oxide, chromium oxide, manganese violet, ultramarine blue, chromium hydrate, and ferric blue. Among the organic pigments, non-limiting mention may be made of carbon black, pigments of D & C type, and lakes based on cochineal carmine or on barium, strontium, calcium or aluminium.

The nacres may be chosen, for example, from: white nacreous pigments, such as mica coated with titanium or with bismuth oxychloride; colored nacreous pigments such as titanium mica with iron oxides; titanium mica with, for example, ferric blue or chromium oxide; titanium mica with, for example, an organic pigment of the abovementioned type; and nacreous pigments based on bismuth oxychloride.

The liposoluble dyes may be chosen from, for example, Sudan Red, D&C Red 17, D&C Green 6, β-carotene, soybean oil, Sudan Brown, D&C Yellow 11, D&C Violet 2, D&C Orange 5, quinoline yellow and annatto. The water-soluble dyes may be chosen from, for example, beetroot juice, methylene blue, the disodium salt of ponceau, the disodium salt of alizarin green, quinoline yellow, the trisodium salt of amaranth, the disodium salt of tartrazine, the monosodium salt of rhodamine, the disodium salt of fuchsin, and xanthophyll.

The composition according to the disclosure may also comprise a filler. The filler may be chosen from fillers that are well known to persons skilled in the art and commonly used in cosmetic compositions. These fillers may be mineral or organic, and lamellar or spherical. Of these fillers, non-limiting mention may be made of talc, mica, silica, kaolin, polyamide powders, such as Nylon® powder (Orgasol® from Atochem), poly-β-alanine powder and polyethylene powder, powders of tetrafluoroethylene polymers, such as Teflon®, lauroyllysine, starch, boron nitride, expanded polymeric hollow microspheres such as those of polyvinylidene chloride/acrylonitrile, for instance Expancel® (Nobel Industrie), acrylic powders, such as Polytrap® (Dow Corning), polymethyl methacrylate particles and silicone resin microbeads (for example Tospearls® from Toshiba), precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms, for example, from 12 to 18 carbon atoms, such as zinc, magnesium or lithium stearate, zinc laurate, and magnesium myristate.

The fillers may be present in the composition of the present disclosure in an amount ranging from 0.1% to 25%, for example, from 1% to 20% by weight, relative to the total weight of the composition.

The composition of the disclosure may also comprise any additive usually used in cosmetics. Non-limiting examples of such additives include, for example antioxidants, preserving agents, fragrances, neutralizers, hydrophilic gelling agents, thickeners, vitamins and fibers, and mixtures thereof.

Needless to say, a person skilled in the art will take care to select the optional additional additives and/or the amount thereof such that the advantageous properties of the composition according to the disclosure are not, or are not substantially, adversely affected by the envisaged addition.

Non-limiting examples of hydrophilic gelling agents that may be used in accordance with the composition of the present disclosure include:

homopolymers or copolymers of acrylic or methacrylic acid or the salts and esters thereof, such as the products sold under the names Versicol F® or Versicol K® by the company Allied Colloid, Ultrahold 8® by the company Ciba-Geigy, and the polyacrylic acids of Synthalen K type;

copolymers of acrylic acid and of acrylamide, such as those sold in the form of a sodium salt under the name Reten® by the company Hercules, sodium polymethacrylate sold under the name Darvan 7® by the company Vanderbilt, and the sodium salts of polyhydroxycarboxylic acids sold under the name Hydagen F® by the company Henkel;

polyacrylic acid/alkyl acrylate copolymers of the Pemulen type;

AMPS (polyacrylamidomethylpropanesulfonic acid partially neutralized with ammonia and highly crosslinked) sold by the company Clariant;

AMPS/acrylamide copolymers of the Sepigel® or Simulgel® type, sold by the company SEPPIC, and

polyoxyethylenated AMPS/alkyl methacrylate copolymers (crosslinked or non-crosslinked),

and mixtures thereof.

The water-soluble film-forming polymers mentioned above may also act as water-soluble gelling agents.

The water-soluble gelling polymer may be present in the composition according to the present disclosure in a solids content ranging from 0.01% to 60% by weight, for example, from 0.5% to 40%, such as from 1% to 30% by weight, for example from 5% to 20% by weight, relative to the total weight of the composition.

The term “fiber” should be understood herein as meaning an object of length L and diameter D such that L is substantially larger than D, wherein D is the diameter of the circle in which the cross section of the fiber is inscribed. In at least one non-limiting embodiment of the present disclosure, fibers having a ratio of length to diameter (L/D) (or shape factor) ranging from 3.5 to 2500, for example, from 5 to 500, such as from 5 to 150, are utilized.

Non-limiting examples of fibers that may be used according to the present disclosure include, for example, fibers having a length ranging from 1 μm to 10 mm, such as from 0.1 mm to 5 mm, for example, from 0.3 mm to 3 mm.

The fibers that may be used in the composition of the disclosure may, for example, be chosen from rigid or non-rigid fibers, and may be of synthetic or natural, mineral or organic origin.

As fibers that may be used in the composition according to the disclosure, non-limiting mention is made of non-rigid fibers such as polyamide (Nylon®) fibers or rigid fibers such as polyimideamide fibers, for instance those sold under the names Kermel® and Kermel Tech® by the company Rhodia or poly(p-phenyleneterephthalamide) (or aramid) fibers sold under the name Kevlar® by the company DuPont de Nemours.

The composition according to the disclosure may be a mascara.

The compositions according to the disclosure may be prepared according to methods known to those skilled in the art.

The composition according to the disclosure may be packaged in a container delimiting at least one compartment that comprises the composition, the container being closed by a closing member.

The container may be associated with an applicator, such as a brush comprising an arrangement of bristles maintained by a twisted wire. Such a twisted brush is described in U.S. Pat. No. 4,887,622. The applicator may also be in the form of a comb comprising a plurality of application members, and may be obtained by molding. Such combs are described, for example, in French Patent No. FR 2 796 529. The applicator may be solidly attached to the container, as described, for example, in French Patent No. FR 2 761 959. In a non-limiting embodiment of the present disclosure, the applicator is attached to a stem, which is itself attached to the closing member.

The closing member may be coupled to the container by screwing. Alternatively, the closing member and the container may be coupled by a means other then screwing, such as via a bayonet mechanism, by click-fastening or by tightening. The term “click-fastening” means any system involving the passing of a rim or bead of material by elastic deformation of a portion, such as of the closing member, followed by return to the elastically unstressed position of the said portion after the rim or bead has been passed.

The container may be at least partly made of thermoplastic material. Non-limiting examples of thermoplastic materials that may be mentioned include polypropylene and polyethylene.

Alternatively, the container may be made of a non-thermoplastic material, such as a glass, a metal, or a metal alloy.

The container may be equipped with a drainer located in the region of the aperture of the container. Such a drainer makes it possible to wipe the applicator and, optionally, the stem to which it may be solidly attached. Such a drainer is described, for example, in French Patent No. 2 792 618.

The contents of any patents or patent applications mentioned above are hereby incorporated by reference into the present patent application.

Other than in the operating examples, or where otherwise indicated, all numbers expressing quantities of ingredients, reaction conditions, and so forth in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches. Also, where a range is given, even if the term “between” is used, the ranges defined include the stated endpoints.

Notwithstanding the numerical ranges and parameters setting forth the broad scope of the invention as approximations, the numerical values set forth in the specific examples are reported as precisely as possible. Any numerical value, however, inherently contains certain errors necessarily resulting from the standard deviation found in its respective testing measurement.

The amounts indicated are weight percentages and are expressed relative to the total weight of the composition, unless otherwise indicated.

The examples that follow serve to illustrate the invention without, however, being limiting in nature.

### EXAMPLE 1

### Mascara

The following mascara was prepared:

| | |
|---|---|
| Hydroxyethylcellulose | 1 |
| Triethanolamine | 3.1 |
| Stearic acid | 5.8 |
| Alcohol wax (Performacol 350 from New | 30 |
| Phase Technologies) | |
| Water | qs 100 |

### EXAMPLES 2 TO 4

### Mascaras

Two mascaras (Examples 3 and 4) according to the disclosure, comprising a fatty alcohol wax (behenyl alcohol) and a cellulose-based polymer, and a mascara according to the prior art (Example 2), comprising a cellulose-based polymer but no fatty alcohol wax, were prepared.

| | Example 2 | | |
|---|---|---|---|
| | (outside the | | |
| | disclosure) | Example 3 | Example 4 |
| Hydroxyethylcellulose | 1 | 1 | 1 |
| Triethanolamine | 3.1 | 3.1 | 3.1 |
| Stearic acid | 5.8 | 5.8 | 5.8 |
| Behenyl alcohol | 0 | 10 | 17 |
| Tacky wax (Kester Wax K82P | 20 | 10 | 3 |
| from Koster Keunen) | | | |
| Water | qs 100 | qs 100 | qs 100 |

For each example, the in vitro charging was measured.

The in vitro charging was measured by gravimetry on two samples of false eyelashes that are curled Caucasian hair (15 hairs about 15 mm long spread over a distance of 1 cm).

The false eyelashes were made up from underneath by effecting 3×10 sweeps of mascara at 2-minute intervals, with uptake of product between each series of 10.

The two samples were dried for 10 minutes at room temperature and then weighed.

The charging at To (C_{To}) of the sample before drying, which corresponds to the charging measured after drying (C_{dry}) divided by the dry extract of the composition (DE), was determined.
C_{To}=C_{dry}/DE

The following results were obtained:

| | Charging |
|---|---|
| | C_{To} (in g) |
| Example 2 | 0.0025 |
| Example 3 | 0.0031 |
| Example 4 | 0.0042 |

The compositions of Examples 3 and 4 according to the present disclosure showed higher charging than a composition not comprising any fatty alcohol wax. It was also found that charging increases as the content of fatty alcohol wax increases.

What is claimed is:

1. A composition for coating keratin fibers comprising, in a cosmetically acceptable aqueous medium, at least 3% of at least one fatty alcohol wax and at least one cellulose-based polymer.

2. A composition for coating keratin fibers comprising, in a cosmetically acceptable aqueous medium, at least one fatty alcohol wax, at least one cellulose-based polymer, and at least one anionic surfactant.

3. The composition of claim 1, wherein the at least one cellulose-based polymer is chosen from alkylcelluloses, hydroxyalkylcelluloses and carboxyalkylcelluloses, and mixtures thereof.

4. The composition of to claim 1, wherein the at least one cellulose-based polymer is present in a solids content ranging from 0.1% to 30% by weight, relative to the total weight of the composition.

5. The composition of claim 4, wherein the at least one cellulose-based polymer is present in a solids content ranging from 1% to 15% by weight, relative to the total weight of the composition.

6. The composition of claim 1, wherein said at least one fatty alcohol wax is chosen from saturated or unsaturated, branched or unbranched fatty alcohols comprising from 14 to 80 carbon atoms, or mixtures comprising at least 30% of the fatty alcohols.

7. The composition of claim 6, wherein said at least one fatty alcohol wax is chosen from linear fatty alcohols comprising from 14 to 60 carbon atoms, branched alcohols comprising from 24 to 80 carbon atoms, and mixtures thereof.

8. The composition of claim 7, wherein said linear fatty alcohols comprise from 20 to 58 carbon atoms.

9. The composition of claim 1, wherein said at least one fatty alcohol wax is present in the composition in an amount ranging from 3% to 50% by weight, relative to the total weight of the composition.

10. The composition of claim 9, wherein said at least one fatty alcohol wax is present in the composition in an amount ranging from 10% to 30% by weight, relative to the total weight of the composition.

11. The composition of claim 1, further comprising at least one additional wax.

12. The composition of claim 11, wherein said at least one additional wax is chosen from hydrocarbon-based waxes, silicone waxes, fluoro waxes, and mixtures thereof.

13. The composition of claim 12, wherein said at least one additional wax is chosen from beeswax, lanolin wax, lemon wax, orange wax, chinese insect waxes, rice bran wax, carnauba wax, candelilla wax, ouricury wax, Japan wax, berry wax, shellac wax, sumach wax, montan wax, microcrystalline waxes, paraffins and ozokerite, polyethylene wax, polymethylene wax, waxes obtained by Fischer-Tropsch synthesis, waxy copolymers, the esters thereof, and combinations thereof.

14. The composition of claim 11, wherein said at least one additional wax has a hardness of less than or equal to 4 MPa.

15. The composition of claim 14, wherein said at least one additional wax has a hardness of less than or equal to 3.5 MPa.

16. The composition of claim 11, wherein said at least one additional wax is chosen from waxes having a tack of greater than or equal to 0.1 N·s.

17. The composition of claim 11, wherein said at least one additional wax is a C₂₀-C₄₀ alkyl(hydroxystearyloxy)stearate.

18. The composition of claim 11, wherein said at least one additional wax is present in the composition in an amount ranging from 5% to 50% by weight, relative to the total weight of the composition.

19. The composition of claim 18, wherein said at least one additional wax is present in the composition in an amount ranging from 10% to 35% by weight, relative to the total weight of the composition.

20. The composition of claim 1, further comprising at least one anionic surfactant.

21. The composition of claim 20, wherein said at least one anionic surfactant is chosen from C₁₆-C₃₀ fatty acid salts; polyoxyethylenated fatty acid salts; phosphoric esters and salts thereof; alkyl ether sulfates; sulfosuccinates; isethionates and acylglutamates, and mixtures thereof.

22. The composition of claim 20, wherein said at least one anionic surfactant comprises at least triethanolamine stearate and/or 2-amino-2-methyl-1,3-propanediol stearate.

23. The composition of claim 20, wherein said at least one anionic surfactant is present in the composition in an amount ranging from 0.01% to 30% by weight, relative to the total weight of the composition.

24. The composition of claim 23, where said at least one anionic surfactant is present in the composition in an amount ranging from 0.1% to 15% by weight, relative to the total weight of the composition.

25. The composition of claim 1, further comprising at least one additional film-forming polymer chosen from synthetic polymers formed by free-radical polymerization, synthetic polymers formed by polycondensation, polymers of natural origin, and mixtures thereof.

26. The composition of claim 25, wherein said at least one additional film-forming polymer is present in the composition in a solids content ranging from 0.1% to 60% by weight relative to the total weight of the composition.

27. The composition of claim 26, wherein said at least one additional film-forming polymer is present in the composition in a solids content ranging from 1% to 30% by weight, relative to the total weight of the composition.

28. The composition of claim 1, wherein the cosmetically acceptable aqueous medium is present in the composition in an amount ranging from 0.1% to 95% by weight, relative to the total weight of the composition.

29. The composition of claim 28, wherein the cosmetically acceptable aqueous medium is present in the composition in an amount ranging from 1% to 80% by weight, relative to the total weight of the composition.

30. The composition of claim 1, further comprising at least one dyestuff.

31. The composition of claim 30, wherein said at least one dyestuff is present in the composition in an amount ranging from 0.01% to 30% by weight, relative to the total weight of the composition.

32. The composition of claim 1, wherein said composition is a mascara.

33. A process for obtaining a charging makeup on keratin fibers and/or obtaining a smooth and uniform deposit on said fibers, said process comprising applying to said fibers a composition comprising, in a cosmetically acceptable aqueous medium, at least 3% of at least one fatty alcohol wax and at least one cellulose-based polymer.

34. The process of claim 33, wherein said keratin fibers are eyelashes or eyebrows.

35. A process for treating keratin fibers, comprising:
applying to said keratin fibers a composition comprising, in a cosmetically acceptable aqueous medium, at least 3% of at least one fatty alcohol wax and at least one cellulose-based polymer.
